(19) United States
(12) Patent Application Publication
(10) Pub. No.: US 2004/0236264 A1
(43) Pub. Date: Nov. 25, 2004

(54) Implantable valve for the treatment of hydrocephalus

(76) Inventor: Alain Lecuyer, Grasse (FR)

Correspondence Address: HARNESS, DICKEY & PIERCE, P.L.C., P.O. BOX 828, BLOOMFIELD HILLS, MI, 48303, US

(21) Appl. No.: 10/798,242

(22) Filed: Mar. 11, 2004

(30) Foreign Application Priority Data
Mar. 12, 2003 (FR) 0303057

(51) Int. Cl. A61M 5/00

(52) U.S. Cl. 604/9

(57) ABSTRACT

The invention concerns a valve which can be implanted in the body of a patient for the treatment of hydrocephalus, comprising:

a housing (7) forming a cavity;

a separation membrane (8) mounted at its periphery on the housing, provided with an orifice (12) and delimiting two chambers in the cavity;

the housing forming in an upstream chamber a seat for the membrane, surrounding the orifice;

a rod (16) with a variable cross-section arranged so as to enter the orifice axially;

means (14, 15) of supporting the rod.

This valve comprises means of axial movement (23) of the means supporting the rod, and drive means (19) for driving the movement means, the drive means being arranged so as to be activated from the outside of the body of the patient so as to allow the adjustment of the valve in a non-invasive manner.

### CROSS REFERENCE TO RELATED APPLICATION

This application claims priority to French Application No. 0303057, filed Mar. 12, 2003, which is incorporated by reference herein.

### BACKGROUND AND SUMMARY

The present invention concerns a valve which can be implanted in the body of a patient for the treatment of hydrocephalus, and more particularly such a valve of the type comprising:

a housing forming a cavity;

a separation membrane mounted at its periphery on the housing, provided with an orifice and delimiting in the said cavity an upstream chamber and a downstream chamber;

the said housing forming in the upstream chamber a seat for the membrane, the said seat surrounding the said orifice;

a rod with a variable cross-section arranged so as to enter the said orifice axially;

means of supporting the rod.

Such valves are already known, in particular through the document FR-A-2 746 659. Valves of this type have in a known fashion a characteristic of differential pressure/flow rate functioning in several zones. When the differential pressure between the upstream chamber and the downstream chamber increases from zero, firstly no flow occurs as long as this differential pressure is insufficient to detach the membrane from its seat. Then, as from a certain differential pressure threshold, the profile of the rod is such that there begins to be a zone in which the differential pressure remains practically constant for a flow rate which increases rapidly. Once a certain flow rate is arrived at a certain flow rate, which is the valve regulation flow rate, the pressure increases for a substantially constant flow rate. Finally, beyond a new differential pressure threshold, the free end of the rod emerges from the orifice in the membrane. The result is a maximum differential pressure, practically independent of the flow rate, which increases on demand.

In known valves, the rod is supported by an element of the housing. Its axial adjustment, which determines the pressure zone with substantially constant flow rate, that is to say the valve regulation flow rate, is carried out definitively at the time of manufacture. When it turns out, after implantation of the valve, that its regulation flow rate is incorrect or is no longer adapted to the state of the patient, it is necessary to remove the valve in order to provide for its replacement. Such an operation is relatively serious and it is therefore desirable to be able to avoid it as far as possible. The invention aims to mitigate this drawback.

More particularly, the aim of the invention is to provide a valve of the type described above which firstly is adjustable after implantation, and secondly where its adjustment can be carried out in a non-invasive fashion. To this end, the object of the invention is a valve which can be implanted in the body of the patient for the treatment of hydrocephalus, of the type comprising:

a housing forming a cavity;

a separation membrane mounted at its periphery on the housing, provided with an orifice and delimiting in the said cavity an upstream chamber and a downstream chamber;

the said housing forming in the upstream chamber a seat for the membrane, the said seat surrounding the said orifice;

a rod with a variable cross-section arranged so as to enter the said orifice axially;

means of supporting the rod;

characterised by the fact that it comprises means of axial movement of the means supporting the rod, and drive means for driving the said movement means, the said drive means being arranged so as to be activated from the outside of the body of the patient so as to allow the adjustment of the valve in a non-invasive manner.

It will be understood that, by moving the rod axially with respect to the plane of the orifice provided in the diaphragm, the differential pressure/flow rate characteristic curve is shifted with respect to the axis of the flow rates. In this way the regulation flow rate of the valve is adjusted. Moreover, this adjustment is carried out in a non-invasive fashion, from the outside of the body of the patient. It can therefore be carried out after implantation of the valve.

In a particular embodiment of the invention, the said rod support means can comprise elastic means. More particularly, the said support means can comprise a plurality of radial elastic support arms fixed at a first one of their ends to the said housing and supporting a rod support sleeve at their other end. Even more particularly, the said first ends of the support arms can be fixed to a fixing ring adjacent to the edge of the said membrane.

Also in a particular embodiment of the invention, the said movement means can comprise a lever mounted on the said housing at a first one of its ends. Also in a particular embodiment of the invention, the said movement means comprise a lever mounted on the said housing and in abutment on the said rod support means, the said lever cooperating with cam means of the said drive means. More particularly, the said lever can comprise an elastic blade fixed to the said housing. In a particular embodiment, the said lever is mounted on the housing at one of its ends, cooperates with the said cam means at its other end, and is in abutment on the said rod support means in its middle part.

Also in a particular embodiment, the longitudinal position of the cam means in the general direction of the lever is adjustable. By adjusting the longitudinal position of the cam means, it is thus possible to adjust the gain in the relationship between the position of the drive means and the axial position of the rod.

It is also possible to provide elastic means for applying the said lever to the said cam means. In one embodiment, these elastic means comprise at least one elastic blade. In the case where the means of fixing the rod comprise elastic arms fixed to a fixing ring, the said elastic blade can be produced in a single piece with the said fixing ring. More particularly, the housing and the fixing ring can comprise projections and complementary recesses for the angular location of the ring with respect to the housing.

In a particular embodiment, the said drive means comprise a magnetic motor. These drive means can also comprise at least one motorised cam arranged so as to cooperate with a cam follower of the said movement means. More particularly, the said motorised cam can comprise a plate arranged so as to be driven in rotation by a motor, the said plate comprising at least two radial ribs of different heights arranged so as to cooperate with the said cam follower.

When the movement means comprise a lever as disclosed previously, the said plate can comprise two pairs of substantially perpendicular radial ribs, the two ribs in each pair being aligned on each side of the axis of rotation of the plate and being substantially of the same height, different from the height of the ribs in the other pair, and the said lever can form, in its part where it cooperates with the same cam, two branches each cooperating with one of the ribs of one of the said pairs of ribs. More particularly, the valve can in this case comprise two elastic blades each in abutment on one of the branches of the said lever.

### BRIEF DESCRIPTION OF THE DRAWINGS

A description will now be given, by way of non-limiting example, of a particular embodiment of the invention, with reference to the accompanying schematic drawings, in which:

FIG. 1 is a view in longitudinal section of a valve according to the invention;

FIG. 2 is a view to a larger scale of the detail 11 in FIG. 1;

FIG. 3 is a perspective view from below of the mechanism of this valve;

FIG. 4 is a perspective view of the valve from below; and

FIG. 5 is a partial view of the mechanism in cross-section and perspective.

### DETAILED DESCRIPTION

The valve 1 in FIG. 1, in fact an adjustable flow rate regulator, is associated here with a non-return valve 2 of a known type, and which will therefore not be described. The fluid enters the valve through a coupling 3 and then passes through the valve and the non-return valve, and is finally drained by a catheter 4. The valve 1 is composed of a part 5 forming a flow rate regulator, and an adjustment part 6. The part 5 is essentially similar to the valve described in the aforementioned document FR-A-2 746 659.

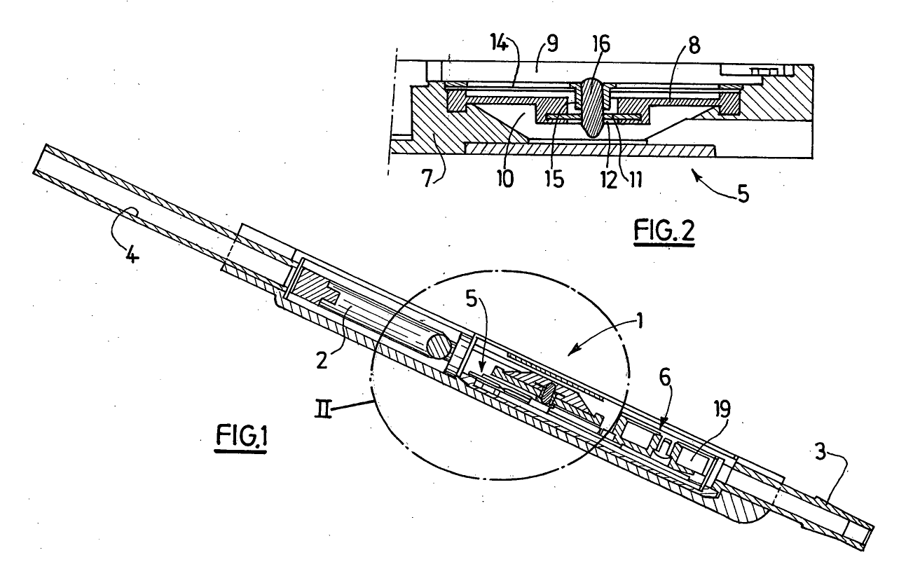

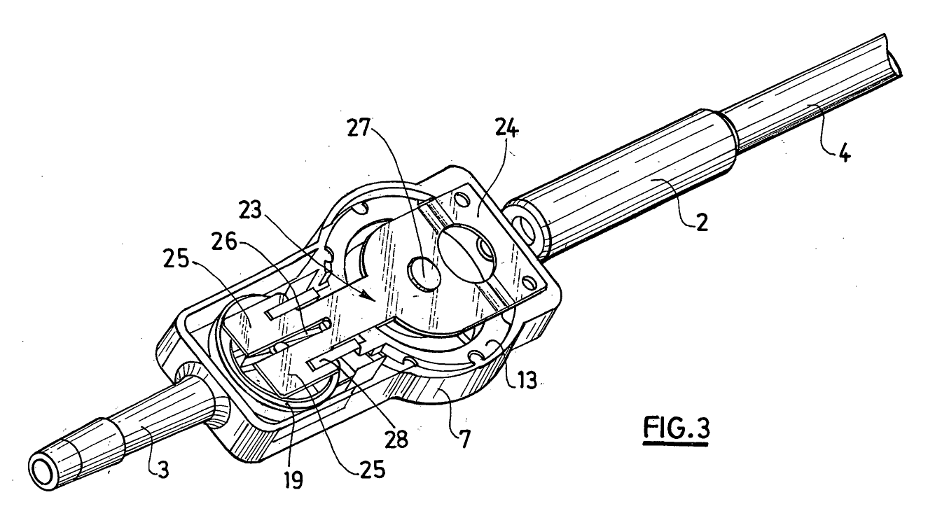

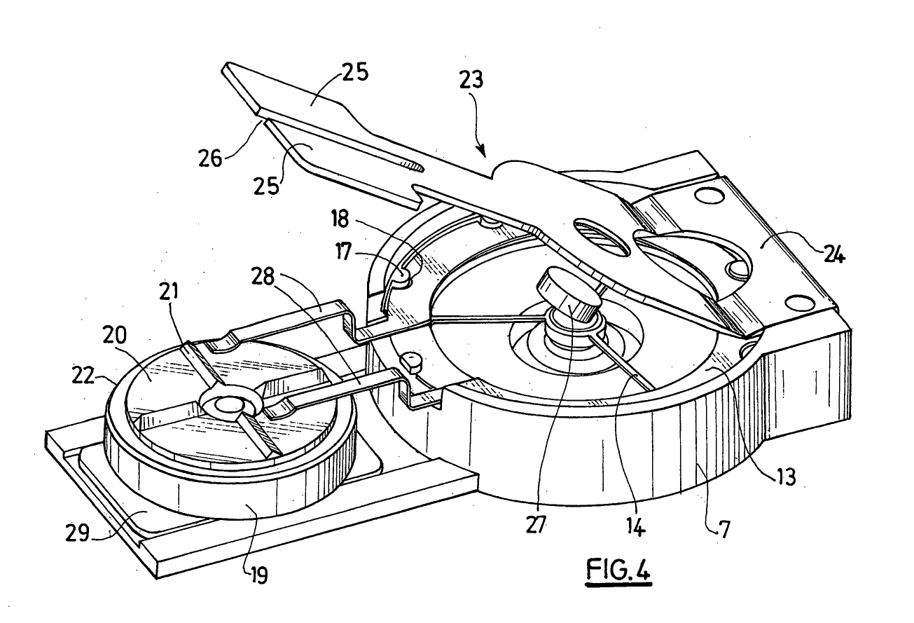

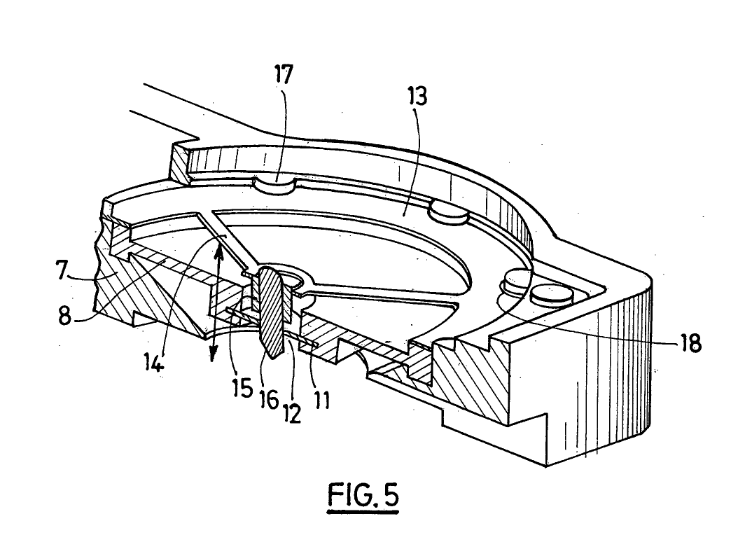

The housing 7 of the valve can be seen in FIG. 2. An elastic membrane 8 mounted at its periphery in the housing 7 delimits in the housing an upstream chamber 9 and a downstream chamber 10. The membrane 8 carries at its centre a washer 11 possessing a central opening 12. The housing forms in the upstream chamber a seat of a known type, not shown.

The outer rim of the membrane 8 is held in the housing 7 simultaneously with a fixing ring 13 which covers this rim. The ring 13 is produced in a single piece with three radial elastic support arms 14 supporting at their internal ends a support sleeve 15 for a rod 16 with a variable cross-section, so that the rod 16 is engaged in the opening 12 in the washer 11. The rod 16 and the edges of the opening 12 therefore determine a cross-section of flow for the fluid, which depends on the degree to which the rod is pushed in since its cross-section is variable. Projections 17 formed in the housing 7 cooperate with complementary recesses 18 formed at the periphery of the ring 13 in order to locate the ring 13 rotationally with respect to the axis of the membrane 8 and of the ring 13.

The valve 1 also comprises a magnetic adjustment motor 19 of any known type. Once the valve is implanted, this motor enables it to be adjusted from outside the body of the patient by means of a suitable magnet. The output shaft of the motor 19 supports a plate 20 in the form of a disc. The plate 20 carries two pairs of radial ribs 21 and 22 oriented at 90° to each other. The two ribs 21 and respectively 22 are symmetrical with respect to the axis of the plate 20. The ribs 22 have a height greater than that of the ribs 21 above the plane of the plate 20.

A lever 23 has one of its ends 24 fixed to the housing 7 on the same side as the part 5 of the valve 1 opposite to the magnetic motor 19. The other end of the lever 23 comprises two branches 25 in the form of vanes, the two branches being separated longitudinally by a slot 26 with a width slightly greater than that of the ribs 21 and 22. In its central part, the lever 23 cooperates with a guidance and support member 27 in order to determine the degree to which the rod 16 is pushed into the washer 11.

Two elastic lugs 28 in a single piece with the ring 13 project from the housing 7, passing above the lever 23, and come into abutment on each of the branches 25 of the lever in order to apply them to two of the ribs 21 or 22 according to the angular position of the plate 20 determined by the magnetic motor 19. The other ribs engage in the slot 26 in the lever 23. Finally, the magnetic motor 19 is disposed on a support plate 29 mounted by any suitable means sliding with respect to the housing 7.

Depending on whether the magnetic motor 19 orients the ribs 21 or the ribs 22 perpendicular to the longitudinal direction of the lever 23, the latter is in abutment on one or other of these ribs. It therefore pushes the rod 16 more or less into the orifice of the washer 11 and thus determines, as disclosed above, the regulation flow rate of the valve 1 from amongst two possible flow rates. The precise adjustment of the value of one or other of these rates is carried out when the valve is assembled by the positioning of the rod 16 in its support sleeve 15.

It is also possible to determine, when the valve is assembled, the separation between the two regulation flow rate values, for two given heights of the ribs 21 and 22. This determination is made by adjusting the sliding of the motor 19 with respect to the housing 7: the closer the motor 19 is to the housing 7, the greater the separation between these values.

1. A valve which can be implanted in the body of the patient for the treatment of hydrocephalus, of the type comprising:
a housing forming a cavity;
a separation membrane mounted at its periphery on the housing, provided with an orifice and delimiting in said cavity an upstream chamber and a downstream chamber;
said housing forming in the upstream chamber a seat for the membrane, said seat surrounding said orifice;
a rod with a variable cross-section arranged so as to enter said orifice axially;
means of supporting the rod;
means of axial movement of the means supporting the rod; and
drive means for driving said movement means;
said drive means being arranged so as to be activated from the outside of the body of the patient so as to allow the adjustment of the valve in a non-invasive manner.

2. A valve according to claim 1, in which said rod support means comprise elastic means.

3. A valve according to claim 2, in which said support means comprise a plurality of radial elastic support arms fixed at a first one of their ends to said housing and supporting a rod support sleeve at their other end.

4. A valve according to claim 3, in which said first ends of the support arms are fixed to a fixing ring adjacent to the edge of said membrane.

5. A valve according to claim 1, in which said movement means comprise a lever mounted on said housing at a first one of its ends.

6. A valve according to claim 1, in which said movement means comprise a lever mounted on said housing and in abutment on said rod support means, said lever cooperating with cam means of said drive means.

7. A valve according to claim 6, in which said lever comprises an elastic blade fixed to said housing.

8. A valve according to claim 6, in which said lever is mounted on the housing at one of its ends, cooperates with said cam means at its other end, and is in abutment on said rod support means in its middle part.

9. A valve according to claim 6, in which the longitudinal position of the cam means in the general direction of the lever is adjustable.

10. A valve according to claim 6, comprising elastic means for applying said lever to said cam means.

11. A valve according to claim 10, in which said elastic means comprise at least one elastic blade.

12. A valve according to claim 11, in which said elastic blade is produced in a single piece with a fixing ring.

13. A valve according to claim 12, in which the housing and the fixing ring comprise projections and complementary recesses for the angular location of the ring with respect to the housing.

14. A valve according to claim 1, in which said drive means comprise a magnetic motor.

15. A valve according to claim 1, in which said drive means comprise at least one motorised cam arranged so as to cooperate with a cam follower of said movement means.

16. A valve according to claim 15, in which said motorised cam comprises a plate arranged so as to be driven in rotation by a motor, said plate comprising at least two radial ribs of different heights arranged so as to cooperate with said cam follower.

17. A valve according to claim 16, in which said plate comprises two pairs of substantially perpendicular radial ribs, the two ribs in each pair being aligned on each side of the rotation axis of the plate and being substantially of the same height, different from the height of the ribs in the other pair, and in which said lever forms, in its part where it cooperates with said cam, two branches each cooperating with one of the ribs in one of said pairs of ribs.

18. A valve according to claim 16, comprising two elastic blades each in abutment on one of branches of a lever of said movement means.

19. A hydrocephalus treatment valve comprising:
a housing including a cavity;
a separation membrane mounted at its periphery on the housing, provided with an orifice and delimiting an upstream chamber and a downstream chamber in the cavity;
a membrane seat in the housing surrounding the orifice;
a rod with a variable cross-section arranged so as to axially enter the orifice;
a rod support being axially movable; and
a driver operably causing the axial movement;
the driver being arranged so as to be activated from outside of a patient and to allow adjustment of the valve in a non-invasive manner.
